(19) United States
(12) Patent Application Publication
(10) Pub. No.: US 2009/0062204 A1
(43) Pub. Date: Mar. 5, 2009

(54) COMPOSITIONS AND METHODS FOR TREATING AND PREVENTING GASTROENTERIC DISEASES

(75) Inventors: Rebecca D. Adkins, Miami, FL (US); Kurt Schesser, Miami, FL (US); Andrea Echeverry, Miami, FL (US)

Correspondence Address: AKERMAN SENTERFITT, P.O. BOX 3188, WEST PALM BEACH, FL, 33402-3188, US

(73) Assignee: UNIVERSITY OF MIAMI, Miami, FL (US)

(21) Appl. No.: 12/202,838

(22) Filed: Sep. 2, 2008

Related U.S. Application Data
(60) Provisional application No. 60/969,383, filed on Aug. 31, 2007.
(60) Provisional application No. 61/037,174, filed on Mar. 17, 2008.

(51) Int. Cl. A61K 38/16; A61K 31/7052; A61P 37/02

(52) U.S. Cl. 514/12; 514/44

(57) ABSTRACT

A model system to study extracellular enteric bacterial pathogen (e.g., Y. enterocolitica) infection of murine neonates through the natural orogastric (o.g.) route of transmission has been developed. The experiments described herein led to the discoveries that 1) murine neonates are resistant to intestinal Yersinia enterolcolitica infection, 2) neonatal mice mount robust Th1, Th2 and Th17 responses to Yersinia infection, 3) mice infected as neonates with Yersinia mount robust antibody responses when re-exposed as adults, 4) mice infected as neonates with Yersinia develop protective immunity, and 5) YopP has different effects in neonates and adults, i.e., while adults show little change in response to a YopP deletant, neonates are more susceptible. Compositions, including adjuvants and vaccines, as well as methods for treating and preventing gastroenteric disease in neonates and children (e.g., humans) and decreasing or preventing inflammation in a neonate or child suffering from an autoimmune disease are described herein.

### CROSS-REFERENCE TO RELATED APPLICATIONS

The present application claims the priority of U.S. provisional application No. 60/969,383 filed on Aug. 31, 2007 and U.S. provisional application No. 61/037,174 filed on Mar. 17, 2008.

### STATEMENT AS TO FEDERALLY SPONSORED RESEARCH

This invention was made with U.S. government support under grant numbers AI44923-02 and AI53459 awarded by the National Institutes of Health. The U.S. government has certain rights in the invention.

### FIELD OF THE INVENTION

The invention relates generally to the fields of medicine, immunology, and microbiology. More particularly, the invention relates to compositions and methods for treating and preventing pediatric gastroenteric disease such as infectious diseases and autoimmune diseases.

### BACKGROUND

Neonates are often susceptible to infectious agents that cause little to no pathology in adults (Adkins et al., Nat. Rev. Immunol. 4:553-564, 2004; Garcia et al., Immunol. Res. 22:177-190, 2000; Holt et al., Allergy 55:688-697, 2000). In particular, neonates are at high risk of developing pathological gastrointestinal infections. Gastrointestinal diseases and their effects cause ˜5 million deaths per year in children. This application focuses on Yersinia enterocolitica, a gram-negative enteric pathogen that causes gastroenteritis, inflammation of the mesenteric lymph nodes (MLN), and in some rare cases, septicemia (Bottone et al., Clin. Microbiol. Rev. 10:257-276, 1997; Naqvi et al., Pediatr Infect Dis J 12:386-389, 1993). Study of this pathogen is particularly relevant to pediatric health since it is considered a prevalent and emerging cause of childhood gastrointestinal disease in the United States (Abdel-Haq et al., J. Pediatr. Infect. Dis. 19:954-958, 2000; Lee et al., J Infect Dis 163:660-663, 1991; Metchock et al., J Clin Microbiol 29:2868-2869, 1991). Indeed, it has been reported that two-thirds of Y. enterocolitica infections occur among infants and children (Black et al., N. Engl. J. Med. 298:76-79, 1978). However, at present, there are few animal systems to model infection of human neonates with enteropathogens. In addition, currently available adjuvants and mucosal vaccines typically elicit weak immune responses. Thus, there is a great need for effective pediatric mucosal vaccines.

### SUMMARY

A model system to study extracellular enteric bacterial pathogen (e.g., Y. enterocolitica) infection of murine neonates through the natural orogastric (o.g.) route of transmission has been developed. The experiments described herein led to the discoveries that 1) murine neonates are resistant to intestinal Yersinia enterolcolitica infection, 2) neonatal mice mount robust Th1, Th2 and Th17 responses to Yersinia infection, 3) mice infected as neonates with Yersinia mount robust antibody responses when re-exposed as adults, 4) mice infected as neonates with Yersinia develop protective immunity, and 5) YopP has different effects in neonates and adults, i.e., while adults show little change in response to a YopP deletant, neonates are more susceptible. Compositions, including adjuvants and vaccines, as well as methods for treating and preventing gastroenteric disease in neonates and children (e.g., humans) and decreasing or preventing inflammation in a neonate or child suffering from an autoimmune disease are described herein.

Accordingly, a composition including an immunologically effective amount of at least one Yop protein or a nucleic acid encoding at least one Yop protein and a pharmaceutically acceptable diluent or carrier is described herein. The at least one Yop protein or nucleic acid encoding at least one Yop protein is an adjuvant. The composition can further include a second adjuvant. The Yop protein can be a Yersinia enterocolitica Yop protein.

Also described herein is a method including administering an immunologically effective amount of a composition including at least one Yop protein or a nucleic acid encoding at least one Yop protein and a pharmaceutically acceptable diluent or carrier to a neonate or child that results in promotion of an immune response in the neonate or child. The composition can be administered mucosally. The neonate or child can be suffering from a bacterial gastroenteric infection. The at least one Yop protein or nucleic acid encoding at least one Yop protein is an adjuvant. The composition can further include a second adjuvant. The Yop protein can be a Yersinia enterocolitica Yop protein.

Further described herein is a method including administering a therapeutically effective amount of a composition including at least one Yop protein or a nucleic acid encoding at least one Yop protein and a pharmaceutically acceptable diluent or carrier to a neonate or child suffering from an autoimmune disease that results in decreasing or preventing inflammation in the neonate or child. The composition can be administered mucosally. The at least one Yop protein or nucleic acid encoding at least one Yop protein is an adjuvant. The composition can further include a second adjuvant. The Yop protein can be a Yersinia enterocolitica Yop protein.

Unless otherwise defined, all technical terms used herein have the same meaning as commonly understood by one of ordinary skill in the art to which this invention belongs.

As used herein, “protein” and “polypeptide” are used synonymously to mean any peptide-linked chain of amino acids, regardless of length or post-translational modification, e.g., glycosylation or phosphorylation.

As used herein, the term “gastroenteric disease” is meant any pathological state of the intestines, of infectious (e.g., bacterial) or non-infectious (e.g., autoimmune colitis) origin.

By the phrases “therapeutically effective amount” and “effective dosage” is meant an amount sufficient to produce a therapeutically (e.g., clinically) desirable result; the exact nature of the result will vary depending on the nature of the disorder being treated. For example, where the disorder to be treated is a gastroenteric disease caused by an enteric pathogen, the result can be promoting an immune response against the pathogen. The compositions described herein can be administered once or several times, e.g., from one or more times per day to one or more times per week, once every several months, etc. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

As used herein, the phrase “an immunologically effective amount” means an amount sufficient to induce an immune response which can prevent bacterial gastroenteric infections or attenuate the severity of any preexisting or subsequent bacterial gastroenteric infections. The exact concentration may be determined by using standard techniques well known to those skilled in the art for assaying the development of an immune response.

As used herein, the term “treatment” is defined as the application or administration of a therapeutic agent described herein, or identified by a method described herein, to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

The terms “patient” “subject” and “individual” are used interchangeably herein, and mean a mammalian subject to be treated, with human patients being preferred. In some cases, the methods of the invention find use in experimental animals, in veterinary applications, and in the development of animal models for disease, including, but not limited to, rodents including mice, rats, and hamsters, as well as primates.

By the term “enteric bacterial pathogen” is meant a disease-causing bacterium that occurs typically in the intestines of humans and other animals.

As used herein, the term “Yop protein” means any Yersinia outer membrane protein essential for pathogenicity. Examples of Yop proteins include Yop A, B, D, E, H, M, O, P, and T. Yop nucleic acid and amino sequences are well known in the art, e.g., accession numbers NP_{—}783702 (yopE), NP_{—}783699 (yopH), NP_{—}783660 (yopM), NP_{—}783657 (yopT), NP_{—}783722 (yopP), NP_{—}783721 (yopO).

Although compositions and methods similar or equivalent to those described herein can be used in the practice or testing of the present invention, suitable compositions and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a series of graphs of survival curves for neonatal and adult BALB/c and C57BL/6 mice after o.g. infection with Y. enterocolitica A127/90 and Y. enterocolitica WA. (A) Seven-day-old () and 7- to 9-week-old (∘) BALB/c mice were infected o.g. with 2×10⁵ CFU Y. enterocolitica A127/90. For each survival curve, data from 9 adult mice and 10 neonatal mice from two independent experiments are depicted. (B) Five C57BL/6 adults and seven neonates were infected o.g. in parallel with 2×10⁶ CFU Y. enterocolitica A127/90. (C) Six neonates and five BALB/c adults were infected o.g. in parallel with 5×10⁶ CFU Y. enterocolitica WA. Survival curves were generated by Kaplan-Meier survival analysis. The rates of death were compared using the Mantel-Haenszel log rank test. *, P=0.0315; ***, P<0.0001.

FIG. 2 is a graph showing survival curves after i.p. infection of neonatal and adult BALB/c mice with Y. enterocolitica A127/90. Seven-day-old () and 7- to 9-week-old (∘) BALB/c mice were infected i.p. with 50 CFU Y. enterocolitica. The curves depict the survival of 12 infected neonates and 9 infected adults. Each curve shows the pooled data from two independent experiments. Survival curves were compared by the log rank test. **, P<0.001.

FIG. 3 is a series of graphs showing colonization kinetics following o.g. infection of neonates and adults with similar doses of Y. enterocolitica A127/90. (A) Seven-day-old () and adult (∘) BALB/c mice were infected o.g. with 5×10⁶ CFU Y. enterocolitica A127/90. On the days indicated, the whole intestine was removed, and bacterial titers were determined. The graph represents the pooled data from three experiments for a total of 5 to 10 neonatal or adult mice. (B to D) At 2, 6, and 7 days p.i., the MLN (B), spleens (C), and livers (D) from mice infected o.g. with 5×10⁶ CFU were collected and processed as described in the text. Each graph depicts the pooled data from three experiments, with a total of 9 to 14 neonatal or adult mice per time point. For all graphs, each symbol is the average titer from duplicate plate counts for an individual mouse. The bold lines represent the mean for each group, and the dashed lines are at the limit of detection for each organ. The Mann-Whitney test was used for analyses comparing neonates and adults on the indicated days. *, P=0.045; **, P=0.0025; ***, P≦0.0004.

FIG. 4 is a series of graphs showing a Pattern of Y. enterocolitica colonization beyond the neonatal gut after o.g. infection with a high dose or when introduced ectopically. Seven-day-old BALB/c mice were infected o.g. with 10× LD₅₀ for neonates (5×10⁷ CFU Y. enterocolitica A127/90). MLN (A), spleens (B) , and livers (B) were collected from infected neonatal mice at 4, 7, and 9 days p.i. and processed as described previously. (C) Neonatal BALB/c mice were infected i.p. with 150 CFU Y. enterocolitica A127/90. The bacterial levels in the spleen and liver were measured at 7 days p.i. in two independent experiments. For all graphs, each symbol represents the titer from an individual mouse. The solid lines show the means, and the dashed lines are at the limit of detection for each organ. *, P=0.0289 for spleens on day 9 compared to spleens on day 7; **, P=0.0046 for MLN on day 7 compared to MLN on day 4.

FIG. 5 is a series of graphs showing distinct anatomical and cellular changes in the MLN of neonates infected with high titers of Y. enterocolitica A127/90. Seven-day-old and adult BALB/c mice were infected o.g. with 5×10⁷ CFU Y. enterocolitica A127/90. (A) MLN from neonates and adults were harvested on different days p.i. and weighed. Controls are age-matched uninfected mice. Data were pooled from three or four independent infections. (B) Individual MLN from neonates and adults were harvested at 3 days p.i., and cell suspensions were stained with anti-Gr-1 and anti-CD68 antibodies. Age-matched uninfected mice served as controls. Fluorescent staining profiles for representative mice are shown. The top box is the neutrophil gate (Gr-1hi CD68int) and the bottom box is the macrophage gate (Gr-1lo-int CD68hi), with their respective percentages indicated.

FIG. 6 is a series of graphs and a pair of photomicrographs showing results from flow cytometric and histological analyses of innate immune cell infiltration into the MLN of neonates infected with different Y. enterocolitica strains. Seven-day-old and adult BALB/c mice were infected o.g. in parallel with 5×10⁷ CFU Y. enterocolitica A127/90 (A, B, and E) or 5×10⁸ CFU Y. enterocolitica WA (C and D). Individual MLN from neonates and adults were harvested 3 days p.i., and cell suspensions were stained with anti-Gr-1 and anti-CD68 antibodies and analyzed as described in the legend to FIG. 5. The percentages of Gr-1hi CD68int cells (neutrophils) are shown in panels A and C, and the percentages of Gr-1lo-int CD68hi cells (macrophages) are shown in panels B and D. The graphs depict the pooled data from two independent experiments for a total of four control and four to six infected mice. Age-matched uninfected mice served as controls. Each symbol represents an individual mouse, with the horizontal lines showing the mean for each group. (E) Individual MLN suspensions from neonates (left) and adults (right) were analyzed by Wright-Giemsa staining. Arrows point to neutrophils in a representative field. Total magnification, ×600. *, P≦0.041; **, P=0.0095 between infected neonates and infected adults.

FIG. 7 is a graph showing bacterial colonization of the neonatal spleen and liver following neutrophil depletion. Neonatal BALB/c mice were injected i.p. with either control IgG or RBC-8C5 (anti-Gr-1) antibody on days −1 and +1 in reference to o.g. infection with 5×10⁷ CFU Y. enterocolitica A127/90. The spleens and livers from all neonates were harvested at 8 days p.i., and bacterial counts were measured as described previously. Each symbol represents an individual mouse, with the lines showing the means from two independent experiments (n=7). The dashed line is at the limit of detection. The number above each group is the percentage of mice with colonized organs. *, P=0.038 between control IgG- and anti-Gr-1-treated neonates.

FIG. 8 is a series of graphs showing survival rates, onset of mortality and bacterial clearance of neonatal and adult mice infected with a low dose of Y. enterocolitica (0.1× LD₅₀). Neonatal BALB/c and adult mice were infected o.g. with a 2×10⁵ CFU Y. enterocolitica WA. A. The survival for each group was monitored for 35 days and mortalities recorded. B. This graph shows that there is a delay in the onset of mortalities in the neonatal group compared to adult mice. **P=0.001 between infected neonates and adults by unpaired-t test C. Fecal pellets were collected from the mice 19 days p.i. and incubated overnight at 4° C. in LB medium for enrichment of Y. enterocolitica. The following day, aliquots of the medium were plated on Yersinia Selective Agar (YSA). The percentage of mice that have cleared Y. enterocolitica is higher (87%) in adult mice compared to 58% in neonatal mice. **P=0.007 between infected neonates and adults by unpaired-t test

FIG. 9 shows the cytokine mRNA profile in the MLN of infected mice. Neonatal BALB/c and adult mice were infected o.g. with 0.1× LD₅₀ of Y. enterocolitica WA. The MLN were harvested 9 days post infection and mRNA extracted from individual mice. The isolated RNA was transcribed into cDNA and tested by real time RT-PCR using Taqman probes against IL-4, IL-17 and IFN-γ. The bar graphs show the fold difference in expression compared to age-matched uninfected mice. *P=0.05 between infected neonates and infected adults by unpaired-t test

FIG. 10 is a series of graphs of inflammatory cytokines produced by CD4+ T cells from the MLN of infected mice. Neonatal BALB/c and adult mice were infected o.g. with 0.1× LD₅₀ of Y. enterocolitica WA. The MLN were harvested 36 days post infection and CD4+ T cells were isolated from 5 neonatal and 3 adult mice. Purified T cells were stimulated with splenic antigen presenting cells pulsed with different concentrations of Heat Killed Yersinia (HKY). Supernatants were harvested 72 hrs after culture and IL-4, IL-17 and IFN-γ quantified by ELISA. Uninfected mice were age-matched with each group (shown in white bars).

FIG. 11 is a quantitative analysis of memory serum antibody responses showing that Yersinia-specific memory antibody responses are comparable between infected neonates and adults. Neonatal BALB/c and adult mice were infected o.g. with 0.1× LD₅₀ Y. enterocolitica WA. 9-11 weeks post primary infection, all the mice were boosted with 2× LD₅₀. Serum was collected 10-21 days post infection and titrated against against the immunogenic LcrV protein in a serum ELISA. IgG1 (top left) and IgG2a (top right) levels were calculated from individual mice and the relative titers compared between neonates and adults.

FIG. 12 are representative antibody profiles from individual mice showing that a greater proportion of infected neonatal mice have an IgG1 biased memory antibody response. Neonatal BALB/c and adult mice were infected as described in FIG. 11. The IgG1/IgG2a profile from 3 individual mice is shown. The percentages of mice (# of positive/total) with high IgG1, high IgG2a or similar IgG1/IgG2a levels are displayed below each profile.

FIG. 13 is a graph showing protective memory response in neonatal mice challenged as adults using weight loss as a marker of disease protection. Neonatal BALB/c and adult mice were infected o.g. with a sublethal dose of Y. enterocolitica WA (0.1× LD₅₀). 10 weeks post primary infection, all the mice were challenged with a lethal dose (50× LD₅₀). Naïve age-matched mice for each group were infected in parallel. Mice were weighed for 15 days and weight loss shown as percent weight over time. *P=0.02 between primed neonates and primed adults by unpaired-t test.

FIG. 14 is a graph showing survival curves of neonatal mice infected with wild type Y. enterocolitica A127/90 and Y. enterocolitica A127/90ΔYopP. 7 day old C57BL/6 were infected o.g. with 2×10⁷ CFU of either strain. Each group of mice was monitored for 25 days and their survival recorded. The survival from 2 independent experiments with 7-8 mice per group was pooled. The time in days that took 50% of mice to succumb (mean time to death) was 16 days for mice infected with wild type Y. enterocolitica and 10 days for mice infected with Y. enterocolitica ΔYopP. The curves were significantly different by Log-rank test **P=0.004.

FIG. 15 is a histological analysis of the small intestine from mice infected with Y. enterocolitica A127/90ΔYopP. 7 day old C57BL/6 mice were infected o.g. with 2×10⁷ CFU of Y. enterocolitica A127/90ΔYopP. 13 days post infection, the whole intestine from infected mice were isolated and fixed in 10% buffered formalin. Tissue was paraffin-embedded and stain with hematoxylin and eosin. Age-matched uninfected mice were used as controls (A). (B) Picture shows inflamed and damaged sections of the small intestine of an infected mouse. Magnification 20×.

FIG. 16 is a histological analysis of the spleen from mice infected with wild type Y. enterocolitica A127/90 and Y. enterocolitica A127/90ΔYopP. 7 day old C57BL/6 mice were infected o.g. with 2×10⁷ CFU of either strain. 13 days post infection, the spleen from uninfected and infected mice were isolated and processed as described in FIG. 15. The pictures show areas of infiltration by immune cells. Magnification 10×.

FIG. 17 is a histological analysis of the livers from mice infected with wild type Y. enterocolitica A127/90 and Y. enterocolitica A127/90ΔYopP. 7 day old C57BL/6 mice were infected o.g. with 2×10⁷ CFU of either strain. 13 days post infection, the liver from uninfected and infected mice were isolated and processed as described in FIG. 15. The pictures show areas of infiltration by immune cells. Magnification 10×.

FIG. 18 is a flow cytometric analysis of the infiltration of granulocytes in the MLN of neonatal mice infected with wild type Y. enterocolitica A127/90 and Y. enterocolitica A127/90ΔYopP. 7 day old C57BL/6 mice were infected o.g. with 2×10⁷ CFU of either strain. Individual MLN were harvested 5 days post infection and cell suspensions were stained with Gr-1 and CD11b antibodies. Age-matched uninfected mice served as controls. Each symbol represents an individual mouse with the line at the mean from 3 independent experiments. **P=0.02 between both groups by unpaired T-test.

FIG. 19 is a graph of the bacterial colonization of the neonatal spleen and liver following infection with wild type Y. enterocolitica A127/90 and Y. enterocolitica A127/90ΔYopP. 7 day old C57BL/6 mice were infected o.g. with 2×10⁷ CFU of either strain. The spleens and livers from all neonates were harvested at 6 days p.i., and bacterial counts were measured as described previously. Each symbol represents an individual mouse, with the lines showing the means from 3 independent experiments (n=8). The dashed line is at the limit of detection. The number above each group is the percentage of mice with colonized organs. *P=0.04 between both groups by unpaired T-test.

### DETAILED DESCRIPTION

Compositions and methods for treating and preventing pediatric gastroenteric diseases caused by a bacterial pathogen by promoting an immune response against the pathogen as well as treating and preventing pediatric autoimmune diseases are described herein. An experimental system of pediatric infection with Yersinia enterocolitica was developed. The results described herein demonstrate that neonates mount robust immune responses to this enteric pathogen. This finding is highly applicable to mucosal vaccinology. The system described herein provides for the identification of potential new adjuvants (Yersinia enterocolitica products) for markedly enhancing the efficacy of pediatric mucosal vaccines. The below described preferred embodiments illustrate adaptations of these compositions and methods. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Immunology techniques are generally known in the art and are described in detail in methodology treatises such as Advances in Immunology, volume 93, ed. Frederick W. Alt, Academic Press, Burlington, Mass., 2007; Making and Using Antibodies: A Practical Handbook, eds. Gary C. Howard and Matthew R. Kaser, CRC Press, Boca Raton, Fla., 2006; Medical Immunology, 6^{th} ed., edited by Gabriel Virella, Informa Healthcare Press, London, England, 2007; and Harlow and Lane ANTIBODIES: A Laboratory Manual, Cold Spring Harbor Laboratory Press, Cold Spring Harbor, N.Y., 1988. Methods involving vaccine techniques are described herein and are generally known in the art. Such methods are described, for example, in methodology treatises including DNA Vaccines (Methods in Molecular Medicine) by Mark W. Saltzman et al., Humana Press, 2006; and New Generation Vaccines by Myron M. Levine et al., Informa Healthcare Press, 2004.

### Enteric Pathogen Infection Model System

Described herein is an enteric pathogen infection model system that can be used to identify a Yop protein(s) that promotes a robust anti-Yersinia immune response in neonates. The identified Yop(s) is used as a novel adjuvant in the formulation of pediatric mucosal vaccines. To best mimic pediatric disease, 7-day-old mice were selected because they are considered to be most reflective of human newborns. Using this system, it was unexpectedly found that neonatal mice are more resistant than adults to primary o.g. infection, as assessed by median lethal dose (LD₅₀) survival experiments. Bacterial colonization experiments revealed that the majority of the bacterial load was contained in the intestines of the neonates, with translocation as far as the MLN but with limited colonization of peripheral organs. However, in adult mice, colonization of deeper tissues (liver and spleen) was observed, which correlated with their susceptibility to lethality. This led to the hypothesis that neonatal mice are competent to mount a strong antibacterial response via enhanced intestinal innate immune responses. Flow cytometric and histological analyses demonstrated that, indeed, neonatal mice exhibited a marked influx of neutrophils and macrophages into the MLN compared to infected adult mice. In addition, depletion of neutrophils by antibody treatment revealed an increase in the translocation of Y. enterocolitica to the spleens and livers of infected neonates. The combined results presented here suggest that neonatal mice may be well equipped to promote a robust intestinal inflammatory response that is highly protective toward at least some types of bacterial enteropathogens. This system can be used to identify a potential adjuvant for a pediatric mucosal vaccine by administering a candidate adjuvant to the Yersinia-infected mice described herein and testing for the mouse's response to the candidate adjuvant (e.g., antibody response, protective immunity, etc.).

### Vaccines and Vaccine Adjuvants

Compositions are described herein that include an immunologically effective amount of at least one Yop protein or a nucleic acid encoding at least one Yop protein and a pharmaceutically acceptable diluent or carrier. In these compositions, the at least one Yop protein or nucleic acid encoding at least one Yop protein generally acts as an adjuvant. Such compositions can include a second adjuvant. Typically, the Yop protein is a Yersinia enterocolitica Yop protein, however any suitable Yersinia species Yop protein can be used. Yersinia products may be used to achieve vigorous vaccine responses to mucosal immunization in children. For example, selected Yersinia proteins (e.g., Yop A, B, D, E, H, M, O, P, T, see Fallman and Gustavson, Int Rev Cytol 246:135-188, 2005; Viboud and Bliska Annu Rev Microbiol. 59:69-89, 2005) can be expressed in recombinant form and extensively purified. Wild-type Yop proteins may be used. Alternatively, genetically engineered derivatives may be used so long as they retain the capacity to elicit robust immune responses in neonates. Examples of derivatives include, but are not limited to, partial Yop proteins or Yop proteins lacking enzymatic activity. These proteins and compositions including these proteins (or nucleic acids encoding these proteins) as described herein can be used as adjuvants for mucosal vaccines—i.e., they can be mixed with recombinant immunogens derived from gastroenteric pathogens commonly affecting children, such as the bacterium Salmonella or rotavirus. In another embodiment, genes encoding Yersinia proteins can be operably linked to genes encoding pathogen proteins and used as a DNA vaccination. Individual Yop proteins as well as a combination of Yop proteins may be used for this purpose. For example, a composition as described herein can include two or more (e.g., two, three, four, etc.) Yop proteins. Similarly, a composition as described herein can include a nucleic acid encoding two or more (e.g., two, three, four, etc.) Yop proteins, or a plurality of nucleic acids, each encoding a different Yop protein.

Vaccine preparation is generally described in Vaccine Design (“The subunit and adjuvant approach” (eds. Powell M. F. & Newman M. J). (1995) Plenum Press New York). In the compositions and methods described herein, any suitable adjuvant may be used. Examples of adjuvants include an aluminum salt such as aluminum hydroxide gel (alum) or aluminum phosphate, but may also be a salt of calcium, iron or zinc, or may be an insoluble suspension of acylated tyrosine, or acylated sugars, cationically or anionically derivatized polysaccharides, or polyphosphazenes. Other known adjuvants include CpG containing oligonucleotides, saponin, cytokines, polyamines, incomplete Freud's adjuvant, and oil emulsions. Additional examples of adjuvants are described in Cox and Coulter (“Animals Parasite Control Utilizing Technology” (ed. Wong, W. K., CRC Press, Boca Raton, Fla., 1992, pages 49-112).

### Gastroenteric and Autoimmune Diseases

Compositions and methods for treating/preventing gastroenteric diseases, including autoimmune diseases, diseases caused by infectious agents, and intestinal inflammation, are described herein. A number of gastroenteric diseases may be treated and/or prevented using the compositions and methods described herein. Examples of gastroenteric diseases include infection by intestinal pathogens (e.g., Shigella, Campylobacter, Helicobacter), autoimmune diseases, and inflammatory bowel diseases. Oral vaccination using Yersinia products may lead to resistance at other mucosal surfaces such as protection against lung infection with influenza virus.

Compositions and Methods for Preventing and Treating Infectious Diseases

Compositions and methods for promoting an immune response in a neonate or child are described herein. In a method of promoting an immune response in a neonate or child (e.g., one suffering from a bacterial gastroenteric infection), an immunologically effective amount of a composition including at least one Yop protein or a nucleic acid encoding at least one Yop protein and a pharmaceutically acceptable carrier or diluent is administered to the neonate or child. In such a method, the at least one Yop protein or nucleic acid encoding a Yop protein is an adjuvant, and the Yop protein is a Yersinia enterocolitica Yop protein.

Compositions and methods for treating/preventing a gastroenteric disease caused by an infectious agent typically involve at least one Yop protein (e.g. Yop P) or a nucleic acid encoding at least one Yop P protein as an adjuvant for enhancing a pediatric vaccine response to the infectious agent. In the experiments described herein, Yersinia was shown to be a potent positive activator of the neonate immune system. In one embodiment of a composition for treating/preventing a gastroenteric disease caused by an infectious agent (e.g., Shigella, Camplylobacter, etc.), the composition includes at least one Yop P protein. In another embodiment of a composition for treating/preventing a gastroenteric disease caused by infectious agent, the composition includes a nucleic acid encoding at least one Yop P protein. For example, a vaccine for preventing a gastroenteeric disease caused by an infectious agent (e.g., Shigella) as described herein includes an immunologically effective amount of a suitable antigen (e.g., a Shigella antigen) and an immunologically effective amount of a composition including at least one Yop P protein or a nucleic acid encoding at least one Yop P protein, and a pharmaceutically acceptable carrier or excipient. The effectiveness of the vaccine can be determined by measuring the anti-Shigella antibody responses in the blood. In addition to a Yop protein or a nucleic acid encoding a Yop protein, compositions as described herein can include a second adjuvant.

In a typical method of preventing or treating a patient (e.g., human child) suffering from or susceptible to a gastroenteric disease, the method includes providing a composition (e.g., a vaccine composition) including at least one (e.g., one, two, three, four, etc.) Yop protein or a nucleic acid encoding at least one Yop protein and administering a therapeutically effective amount of the composition to the patient.

### Compositions and Methods for Preventing and Treating Autoimmune Diseases

In some embodiments, compositions and methods described herein can be used to treat or prevent an autoimmune disease in adult and pediatric mammalian subjects (e.g., humans). For example, compositions and methods as described herein may be used to modulate (e.g., downregulate) intestinal inflammation in a subject (e.g., human child) having inflammatory bowel disease (e.g., pediatric inflammatory bowel disease (PIBD)). In one embodiment of a composition for treating intestinal inflammation in a pediatric subject having PIBD, for example, the composition includes purified Yop P (e.g., purified recombinant Yop P) in a pharmaceutically acceptable vehicle. Such a composition would typically be administered orally to the pediatric subject resulting in a reduction or elimination of intestinal inflammation. In another embodiment of a composition for treating intestinal inflammation in a pediatric subject having PIBD, for example, the composition includes a nucleic acid encoding at least one Yop protein (e.g., Yop P) in a pharmaceutically acceptable vehicle.

Any of the compositions described herein can further include, for example, carriers, excipients, transfection facilitating agents, and/or adjuvants as described herein. Compositions of the present invention may include various salts, excipients, delivery vehicles and/or auxiliary agents as are disclosed, e.g., in Remington: The Science and Practice of Pharmacy, 19^{th} ed., Mack Publishing Co., 1995, which is incorporated herein by reference in its entirety. A vaccine as described herein might be packaged in various forms, including packaging in a liquid, gel, or solid form that may be a tablet or gelcap or a component of a food carrier material, such as a pudding or yogurt.

### Administration of Compositions

The compositions of the invention may be administered to neonates and children (e.g., rodents, humans) in any suitable formulation. For example, one or more Yop proteins or a nucleic acid encoding one or more Yop proteins may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to neonates and children by any conventional technique. Typically, such administration will be mucosal, but administration can also be parenteral (e.g., intravenous, subcutaneous, intratumoral, intramuscular, intraperitoneal, or intrathecal introduction). The compositions may also be administered directly to a target site by, for example, surgical delivery to an internal target site or by catheter. The compositions may be administered in a single bolus, multiple injections, or by continuous infusion (e.g., intravenously, by peritoneal dialysis, pump infusion). For parenteral administration, the compositions are preferably formulated in a sterilized pyrogen-free form.

### Effective Doses

The compositions described above are preferably administered to a neonate or child (e.g., a rat, human) in an effective amount, that is, an amount capable of producing a desirable result in a treated mammal (e.g., reducing inflammation in the gastrointestinal tract of a mammal suffering from or susceptible to an autoimmune or promoting an immune response in a mammal suffering from or susceptible to a bacterial gastroenteric infection). Such a therapeutically effective amount can be determined as described below.

Toxicity and therapeutic efficacy of the compositions utilized in compositions and methods of the invention can be determined by standard pharmaceutical procedures, using either cells in culture or experimental animals to determine the LD₅₀ (the dose lethal to 50% of the population). The dose ratio between toxic and therapeutic effects is the therapeutic index and it can be expressed as the ratio LD₅₀/ED₅₀. Those compositions that exhibit large therapeutic indices are preferred. While those that exhibit toxic side effects may be used, care should be taken to design a delivery system that minimizes the potential damage of such side effects. The dosage of preferred compositions lies preferably within a range that includes an ED₅₀ with little or no toxicity. The dosage may vary within this range depending upon the dosage form employed and the route of administration utilized.

As is well known in the medical and veterinary arts, dosage for any one subject depends on many factors, including the subject's size, body surface area, age, the particular composition to be administered, time and route of administration, general health, and other drugs being administered concurrently.

### EXAMPLES

The present invention is further illustrated by the following specific examples. The examples are provided for illustration only and should not be construed as limiting the scope of the invention in any way.

### Example 1

### Murine Neonates are Highly Resistant to Yersinia enterocolitica Following Orogastric Exposure

Neonates are considered highly susceptible to gastrointestinal infections. This susceptibility has been attributed partially to immaturity in immune cell function. To study this phenomenon, a model system with murine neonates was developed, using the natural orogastric route of transmission for the enteropathogen Yersinia enterocolitica. The susceptibilities of 7-day-old and adult mice to orogastric Y. enterocolitica infection were assessed in 50% lethal dose experiments. Remarkably, neonatal mice of either the BALB/c or C57BL/6 mouse strain showed markedly enhanced survival after infection compared to adult mice. The resistance of neonates was not due to failure of the bacteria to colonize neonatal tissues; Y. enterocolitica was readily detectable in the intestine and mesenteric lymph nodes (MLN) for at least 1 week after infection. In adult mice, Y. enterocolitica rapidly disseminated to the spleen and liver. In striking contrast, bacterial invasion of the spleen and liver in neonates was limited. Using flow cytometry and histology, substantial increases were observed in the percentages of neutrophils and macrophages in the neonatal MLN, while influx of these cells into the adult MLN was limited. Similar results were obtained using two different high-virulence Y. enterocolitica strains. Importantly, depletion of neutrophils with a specific antibody led to increased translocation of the bacteria to the spleens and livers of neonates. Together, these experiments support the hypothesis that the neonatal intestinal immune system can rapidly mobilize innate phagocytes and thereby confine the bacterial infection to the gut, resulting in a high level of resistance.

### Material and Methods

Wild-type high-virulence Y. enterocolitica A127/90 serotype 0:8/biotype IB and Y. enterocolitica WA serotype 0:8/biotype IB were used in this study. Prior to any experiments, the Y. enterocolitica WA strain was passaged in vivo to maintain virulence by infecting C57BL/6 mice o.g. The spleen of an infected mouse was homogenized in Ca²⁺—Mg²⁺ Hanks' balanced salt solution (HBSS), and diluted aliquots were plated on Yersinia-selective agar (YSA) plates (Difco, Sparks, Md.). For both strains, bacterial stocks were prepared by overnight culture of fresh bacterial colonies in Luria-Bertani (LB) broth (EMD, San Diego, Calif.) at 27° C. Cultures were diluted 1:20 in LB broth the next day and grown to early stationary phase. Each strain was stored in 1-ml aliquots in 30% glycerol-LB at −80° C. The titer of the frozen stock was determined by plating a diluted aliquot on LB plates and then counting colonies after incubation for 48 h at 27° C.

BALB/c (Charles River, Wilmington, Mass.) and C57BL/6 (The Jackson Laboratory, Bar Harbor, Me.) mice were bred and housed under barrier conditions in the Division of Veterinary Resources of the University of Miami Miller School of Medicine. Mice were confirmed to be free of common infectious agents through periodic colony screenings. Adult female mice (7 to 9 weeks of age) and neonatal mice (7 days of age) were used in all experiments. Adult mice were given sterile food and water ad libitu prior to infection. Neonatal mice from different litters were separated from the dams and randomly mixed prior to infection. Immediately after infection, the neonates were returned to the dams. Neonatal mice were nursing throughout all the experiments.

Bacterial frozen stocks were washed twice with HBSS and diluted in consecutive 10-fold dilutions to the desired concentrations in sterile filtered 0.1% blue food coloring (McCormick, Baltimore, Md.) in HBSS. Blue food coloring was used to facilitate visualization of delivery of the inoculum to the neonatal stomach. For LD₅₀ analyses, groups of four or five adult mice per dose were inoculated o.g. with the bacterial suspensions via a 1-in.-long, 22-gauge, round-tipped feeding needle (Fine Science Tools, Foster City, Calif.) attached to a 1-ml syringe. Five to seven neonates were intubated o.g. with PE-10 tubing (polyethylene tube with an outside diameter of 0.61 mm; Clay Adams, Sparks, Md.) attached to a 30-gauge needle and a Hamilton syringe. Adults received 100 μl and neonates received 25 μl of 10-fold dilutions of bacterial suspensions ranging from 2×10³ to 2×10⁸ CFU of Y. enterocolitica A127/90, depending on the experiment, or from 5×10⁵ to 5×10⁷ CFU of Y. enterocolitica WA. Mice were excluded from all experiments when signs of inoculum aspiration were visible, including sneezing and excess fluid in the mouth and nostrils. For infections by the intraperitoneal (i.p.) route, neonatal and adult mice were infected i.p. in parallel, using 30- and 25-gauge needles, respectively. Doses ranged from 10 to 200 CFU Y. enterocolitica A127/90 in a 50-μl volume. For all experiments, the actual administered dose was determined by plating serial dilutions of the suspensions on LB plates and incubating them at 27° C. for 48 h. For each experiment, there were four or five adult mice and six or seven neonatal mice per dose. The LD₅₀ means were calculated from three independent experiments. All mice were monitored for signs of distress twice daily for 21 days; mice that had become unable to move were euthanized, and their deaths were included in the analysis.

Bacterial enumeration from organs of infected mice. To measure Yersinia titers in whole gut tissue after o.g. infection, the intestines were excised, from 0.5 cm below the pylorus to about 0.5 cm above the rectum, and placed intact into cold HBSS. The intestines were weighed and homogenized in 5 to 10 ml of cold HBSS, using a Seward Biomaster 80 stomacher (Brinkmann, Westbury, N.Y.) for 2 to 3 min at high speed. The MLN, spleens, and livers of infected mice were excised sterilely at 2 to 9 days postinfection (p.i.) and were homogenized for 2 to 3 min at medium speed in 3 to 6 ml or 3 to 10 ml of cold HBSS, for neonatal and adult organs, respectively. Viable duplicate plate counts were done by spreading serial dilutions of the suspensions on YSA plates and incubating them at 27° C. for 48 h. Control experiments with age-matched uninfected mice demonstrated that intestinal commensal bacteria were undetectable using this selective medium; therefore, it was assumed that all bacterial colonies were indeed Y. enterocolitica. The average titer was calculated and expressed as log CFU per gram of tissue. The lower limit of detection for the assay was estimated using similar calculations, assuming the presence of one bacterial colony in the lowest dilution. The limit of detection for all organs of infected neonates and adults ranged from 1.78 to 2.94 log CFU/g tissue.

Neonates and adults were infected o.g. in parallel with Y. enterocolitica A127/90 or Y. enterocolitica WA. Individual Peyer's patches (PP) from adults and MLN and spleens from both groups were harvested at 3 and 5 days p.i. and placed in cold HBSS containing 1% calf serum (Gibco), 10 mM HEPES (Gibco), and 4 mM sodium azide. Age-matched uninfected mice served as controls. For neutrophil and macrophage analysis, 5×10⁵ to 1×10⁶ cells were incubated in mouse Fc Block (CD16/CD32; BD Pharmingen, San Diego, Calif.) for 5 min on ice, followed by a 30-min incubation with phycoerythrin-conjugated anti-Gr-1 (Ly6C/Ly6G) (BD Pharmingen). To detect intracellular CD68 expression, the cells were fixed, permeabilized, and stained with Alexa Fluor 647-conjugated anti-CD68 antibody (FA-11) per the manufacturer's instructions (Serotec, Raleigh, N.C.). Samples were analyzed on a Becton Dickinson LSR I flow cytometer. Neutrophils were characterized as Gr-1hi CD68int cells, and macrophages were characterized as Gr-1lo-int CD68hi cells.

In parallel to the flow cytometric studies, MLN suspensions from infected neonatal and adult mice were used for histological identification of neutrophils. Age-matched uninfected mice served as controls. Cells (5×10⁴) were spun onto positively charged slides (VWR, West Chester, Pa.) at 800 rpm for 8 min, using a cytocentrifuge. Slides were allowed to air dry for 1 h, followed by fixation in 100% methanol. Dry slides were submerged in Wright stain (Sigma, St. Louis, Mo.) for 6 min, followed by soaking in Sorensen buffer (0.15 M Na2HPO4, 0.15 M KH2PO4, pH 6.5) for 3 min. Finally, slides were stained with Giemsa stain (Sigma) for 4 min and washed with distilled water. All cells were viewed under a Leitz Laborlux S microscope at a magnification of ×630.

RBC-8C5 antibody (anti-Gr-1) was collected from ascites fluids grown in nu/nu mice and purified over a protein G Sepharose column (GE Healthcare, Piscataway, N.J.). Seven-day-old BALB/c mice were injected i.p. with either control rat IgG antibody (Jackson ImmunoResearch Inc., West Grove, Pa.) or RBC-8C5 antibody 1 day before and 1 day after o.g. infection with 5×10⁷ CFU Y. enterocolitica A127/90. All mice received a total of 200 μg of the appropriate antibody in 50-μl injections. The whole intestine, MLN, spleen, and liver were collected at 8 days p.i. and processed as previously described for bacterial counts. To ensure that neutrophils had been depleted, flow cytometric analysis of individual MLN and spleen cells was performed after antibody treatment and o.g. infection. Neutrophils were characterized as Gr-1hi CD68int cells and found to be reduced to ≦0.02% of the cells in each tissue.

Statistical analysis. LD₅₀ values were estimated using the method of Reed and Muench (Am. J. Hyg. 27:493-497, 1938). Survival curves were generated by the Kaplan-Meier method, and survival kinetics between neonatal and adult groups were analyzed by the Mantel-Haenszel log rank test (GraphPad Prism 4). Survival curves were considered significantly different when the P value was ≦0.05. For colonization experiments, the data from two or three independent experiments were pooled before analysis. Flow cytometric and histological analyses were performed twice for each Y. enterocolitica strain. The means between groups were analyzed by the Mann-Whitney test, with significance for P values of ≦0.05. Neutrophil depletion was performed twice, and the mean bacterial titers were analyzed by an unpaired t test, with significance for P values of ≦0.05, after confirming that each group followed a normal distribution.

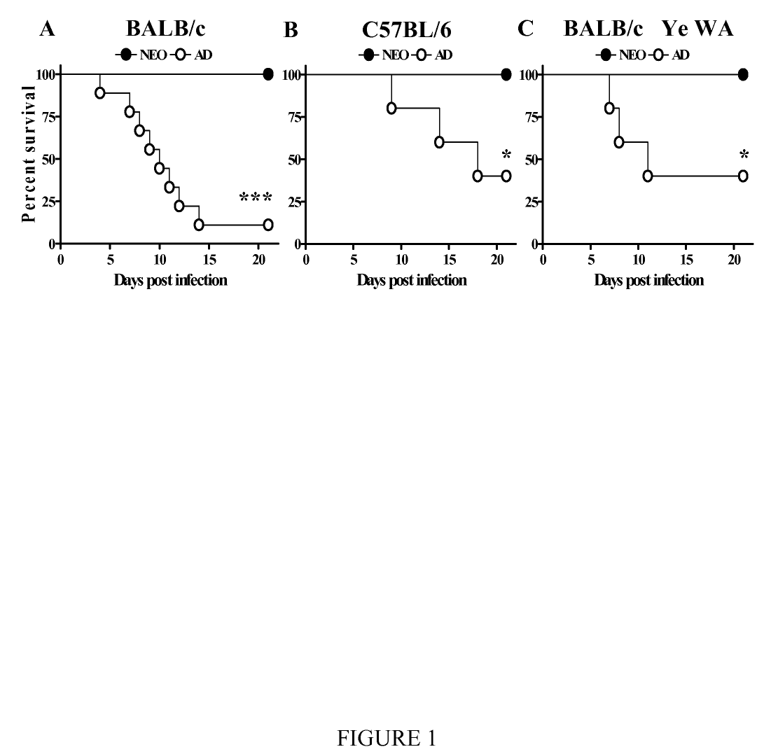

Neonatal mice are highly resistant to infection with Y. enterocolitica via the natural (o.g.) route of exposure. Since the susceptibility of neonatal mice to Y. enterocolitica has not been reported, infections in neonates and adults were compared. For this purpose, 7-day-old and adult BALB/c and C57BL/6 mice were infected, using the natural (o.g.) route of transmission. LD₅₀ survival experiments with the high-virulence Y. enterocolitica strain A127/90 (biotype IB/serotype 0:8) were performed. Surprisingly, it was found that neonatal mice were more resistant than adult mice to lethal o.g. Y. enterocolitica infection (Table 1). The differences in the average LD₅₀ values between neonates and adults of the BALB/c and C57BL/6 strains were approximately 50- and 46-fold, respectively. Comparison of the geometric means between groups showed similar differences (38- and 61-fold for BALB/c and C57BL/6 mice, respectively) (Table 1). These results indicated that the patterns of resistance for both mouse strains were similar in that 7-day-old mice were highly resistant to o.g. infection compared to adult mice. This is clearly demonstrated in FIGS. 1A and B, which show significant differences in the survival rates of neonates and adults of both mouse strains (BALB/c [P<0.0001] and C57BL/6 [P=0.0315]) infected with this isolate of Y. enterocolitica.

TABLE 1
LD₅₀ (CFU)

| Mouse | | | | | Geometric | |
|---|---|---|---|---|---|---|
| strain | Group | Expt 1 | Expt 2 | Expt 3 | mean LD₅₀^{c} | Avg LD₅₀ |
| BALB/c | Neonates | 2.7 × 10⁵ | 1.9 × 10^{7b} | 2.7 × 10⁵ | 1.1 × 10⁶ | 6.5 × 10⁶ |
| | Adults | 8.2 × 10³ | 3.8 × 10^{5b} | 7.8 × 10³ | 2.9 × 10⁴ | 1.3 × 10⁵ |
| C57BL/6 | Neonates | 2.4 × 10⁶ | 1.7 × 10⁶ | 1.5 × 10^{8b} | 8.5 × 10⁶ | 5.1 × 10⁷ |
| | Adults | 2.3 × 10⁵ | 3.4 × 10⁴ | 3.3 × 10^{6b} | 1.4 × 10⁵ | 1.2 × 10⁶ |

Average LD₅₀ values after o.g. infection of BALB/c and C57BL/6 neonatal and adult mice with Y. enterocolitica A127/90^{a}
^{a}Mice were infected o.g. with 10-fold dilutions of bacterial suspensions ranging from 2 × 10³ to 2 × 10⁸ CFU.
^{b}LD₅₀ values were derived from infections performed in parallel for each mouse strain.
^{c}The geometric mean was derived by calculating the mean of the logarithms of the values and then calculating the antilog of the mean.

The Y. enterocolitica strain A127/90 is a clinical isolate which has been used by other researchers for multiple in vitro characterizations. However, this is the first time that the LD₅₀ for the A127/90 strain has been reported in the literature. The LD₅₀ value we observed for the A127/90 strain in adult mice was nearly a log lower than the values reported for other strains of Y. enterocolitica. Therefore, to ensure the universality of the findings, an o.g. LD₅₀ experiment was performed using another high-virulence strain, Y. enterocolitica WA (biotype IB/serotype 0:8). The LD₅₀ value we obtained for adult BALB/c mice with Y. enterocolitica WA (3×10⁶ CFU) was similar to that previously reported in the literature. Furthermore, as found for the A127/90 strain, neonatal BALB/c mice were also more resistant to o.g. infection with the WA strain; the LD₅₀ was still at least a log higher for neonates (5×10⁷ CFU) than for adults (3×10⁶ CFU). The proportions of neonates and adults surviving infection with the same dose of Y. enterocolitica WA were also significantly different (P=0.0315) (FIG. 1C). Although the difference (17-fold) in o.g. LD₅₀ values between BALB/c neonates and adults after Y. enterocolitica WA infection was not as high as that for Y. enterocolitica A127/90 (50-fold), this result extends and supports the conclusion that neonatal mice are highly resistant to o.g. Y. enterocolitica exposure.

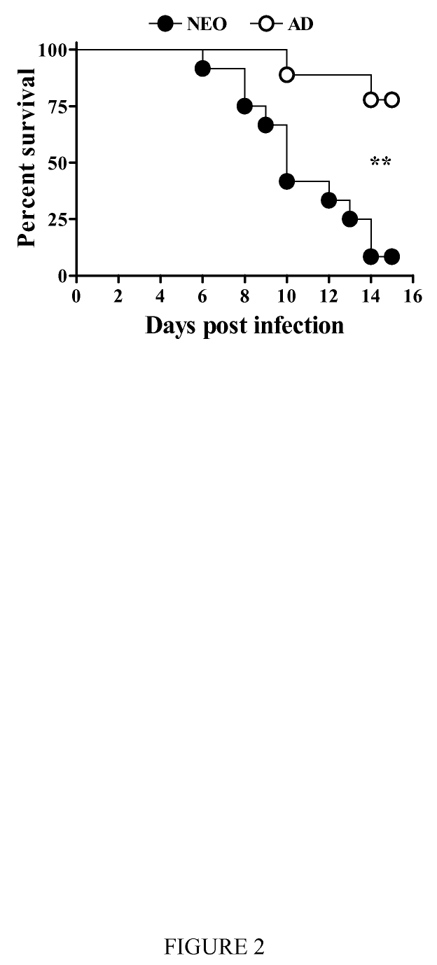

Neonatal mice are very susceptible to ectopic (i.p.) infection with Y. enterocolitica. The finding that neonatal mice showed increased survival after o.g. infection was surprising, since neonates are generally thought to be more susceptible to bacterial infections. To determine if the resistance of neonates was restricted to the o.g. route of administration, survival experiments were carried out using Y. enterocolitica A127/90 injected i.p. into BALB/c mice. The results, shown in Table 2, revealed that neonates were at least one-half a log more susceptible to Y. enterocolitica by i.p. infection than were adult mice; the neonatal survival rate after i.p. injection was also significantly (P<0.001) decreased compared to that for adults (FIG. 2). These findings indicated that in contrast to the case for o.g. infection, neonates could not efficiently control infection when bacteria were administered ectopically. Note that the difference in LD₅₀ values between o.g. and i.p. infections was quantitatively greater for neonates (>200,000 times) than it was for adults (>700 times). These results suggest that Y. enterocolitica administered through the natural route triggers a local response in neonates that is highly protective, in contrast to what occurs after i.p. infection. Together, the results from these survival experiments demonstrate that 7-day-old mice are capable of efficiently controlling infection with this pathogen only if it is encountered through the natural, o.g. route of exposure.

TABLE 2
LD₅₀ (CFU)

| Group | Expt 1 | Expt 2 | Expt 3 | Avg LD₅₀ |
|---|---|---|---|---|
| Neonates | 19 | 21 | 40 | 30 |
| Adults | 138 | 189 | 178 | 168 |

Average LD₅₀ values after i.p. infection of neonatal and adult BALB/c mice with Y. enterocolitica A127/90^{a}
^{a}Mice were infected i.p. in parallel with 50 μl of Y. enterocolitica A127/90 suspensions ranging from 10 to 200 CFU.

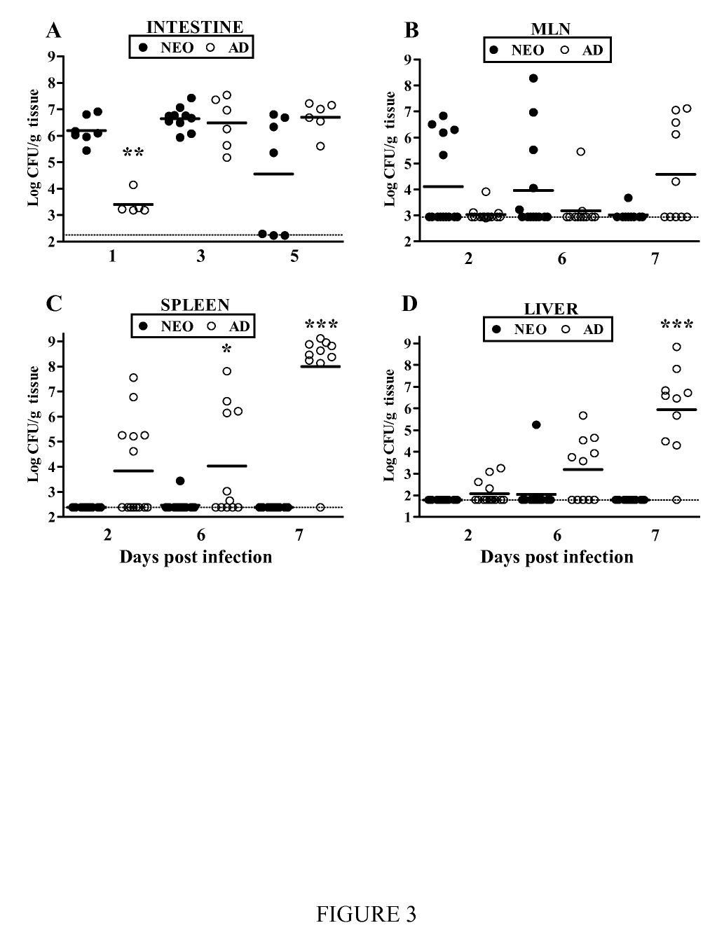

Following o.g. infection, Y. enterocolitica is largely confined to the intestinal tissues of neonates. The enhanced resistance of neonates to Y. enterocolitica could potentially be due to developmental or physiological differences between neonates and adults. For example, an immature intestinal environment might preclude efficient colonization of the neonatal gut, leading to rapid clearance of the bacterium from the body. To investigate this possibility, kinetic studies of bacterial colonization of the intestines were performed. Initially, colonization of the whole intestine in neonates and adults infected with the same dose (5×10⁶ CFU) of Y. enterocolitica was examined. This dose corresponds to approximately 0.8× LD₅₀ for neonates and 40× LD₅₀ for adults. The adult intestine harbored low but detectable levels of bacteria at 1 day p.i. (FIG. 3A). In contrast, high levels of Y. enterocolitica were detected in all of the neonatal mice 1 day after infection and persisted for at least the first 3 days p.i. By this time point, the mean titers for both groups were not significantly different, and at 5 days p.i., high titers were detected in 100% of adult mice, in contrast to 57% of neonatal mice. These data indicate that there is high-level colonization of the neonatal intestine immediately after inoculation and that the enhanced resistance of neonates is not caused by prompt elimination of the infectious inoculum.

Next, whether or not the enhanced resistance of neonates after o.g. infection was due to failure of the bacteria to invade past the intestinal lumen to establish systemic infection was addressed. For this purpose, the colonization kinetics of the MLN, spleen, and liver was compared in neonates and adults during the first week after infection. Y. enterocolitica was detected as early as 2 days p.i. in the MLN of 36% of infected neonates, in contrast to 7% of infected adults (FIG. 3B). These relative titers were maintained in at least 38% of the neonatal mice analyzed 6 days after infection. By 7 days p.i., the pattern was reversed, with a greater proportion of adult mice (50%) having detectable Y. enterocolitica in the MLN, in contrast to 11% of neonates. In contrast, the relative colonization of the spleen and liver followed a completely different pattern. Viable bacteria were readily detected at 2 days p.i. in the spleens (FIG. 3C) and livers (FIG. 3D) of 43% and 28% of infected adult mice, respectively. At this time point, the bacterial titers were already high in the spleen. By 6 days p.i., at least 54% of the adult mice showed high titers in the spleen and liver. These titers increased significantly in both organs by 7 days p.i. (P<0.0048) (FIGS. 3C and D). In striking contrast, Y. enterocolitica was detectable only in the spleen (FIG. 3C) and liver (FIG. 3D) of 1 of 36 infected neonates over the entire 7-day period of analysis. Note that the high bacterial levels in the adult spleen and liver correlate with their susceptibility to the lethal effects of the infection. Together, these findings show that Y. enterocolitica is able to efficiently colonize the intestinal tissues of adults and neonates but that further dissemination of bacteria beyond the gut is limited in neonates.

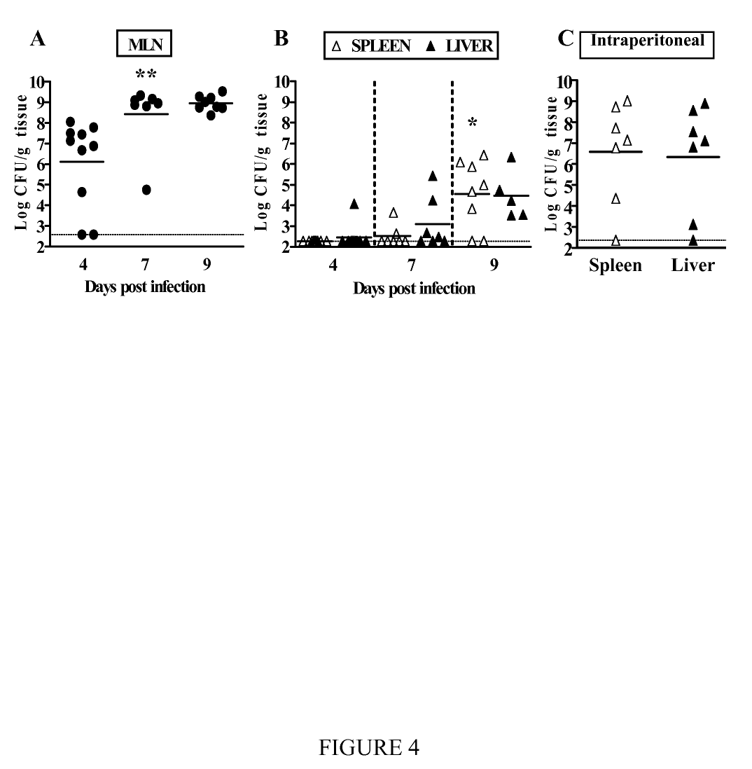

The observation that Y. enterocolitica is largely retained in the guts of neonates offers a potential explanation for their high-level resistance, since containment of the bacteria in the intestine or MLN could potentially spare vital organs from the harmful effects of an inflammatory response. However, this result also raised the possibility that the lack of detectable bacteria in the spleen was due to developmental immaturity in the neonate that prevented systemic colonization. Therefore, whether it was possible to colonize the neonatal spleen and liver with Y. enterocolitica under any conditions of infection was determined. Initially, this question was addressed by increasing the infectious dose given o.g. to neonates. As shown in FIG. 4A, once the titer of bacteria was increased to 10× LD₅₀ for neonates (5×10⁷ CFU), colonization of the MLN was evident at 4 days p.i., when 80% of infected neonates had substantial bacterial titers in the MLN. The bacterial levels in the neonatal MLN increased significantly (P=0.0046), by 2 log, 3 days later and persisted at high numbers as late as 9 days p.i. in 100% of analyzed mice. In contrast, viable Y. enterocolitica was not detectable in the neonatal spleen at 4 days p.i., even in the presence of high bacterial titers in the MLN (FIG. 4B). Only one neonate had detectable bacteria in the liver at this time point. By 7 days p.i., it was possible to recover Y. enterocolitica from the spleens and livers of 28.6% and 57% of infected neonates, respectively. However, the average levels of bacteria in these tissues were reduced over 4 log compared with the titers found in the MLN at the same time point. By 9 days p.i., greater percentages of mice had higher but comparable bacterial levels in the spleen (75%) and liver (100%). Interestingly, the mean Y. enterocolitica level found in the liver at 9 days p.i. was at least 1 log lower than that detected in the adult liver 7 days after infection with a comparable dose (FIG. 3D). Strikingly, the mean bacterial level detected in the neonatal spleen (4.6 log) at 9 days p.i. was at least 3 log lower than that found in the adult spleen (8.0 log) after 7 days of infection (FIG. 3C). This may indicate that despite translocation of the bacteria to peripheral tissues, neonates carry lower titers than infected adult mice.

To demonstrate independently the ability of Y. enterocolitica to spread to deeper tissues following infection, colonization experiments were done using another route of infection. When neonatal mice were infected i.p. with 5×LD₅₀ (150 CFU), a substantial colonization of the spleen and liver was observed at 7 days p.i. in 86% of mice analyzed (FIG. 4C). Collectively, these data indicate that Y. enterocolitica is efficiently confined to the intestinal tissues upon o.g. exposure and when the infective doses are sublethal. However, some systemic spread can be observed when neonates are exposed to lethal bacterial doses o.g. or when the bacteria are introduced ectopically.

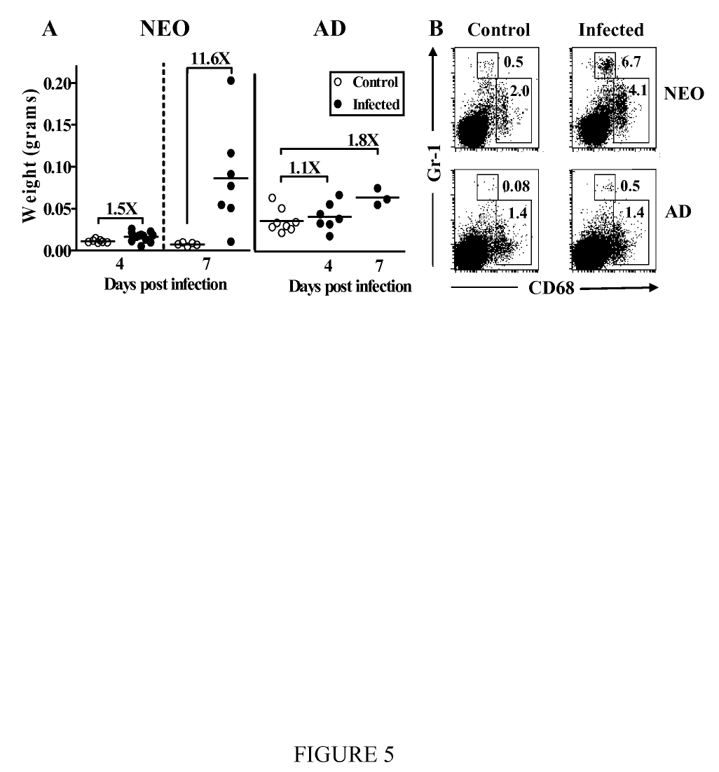

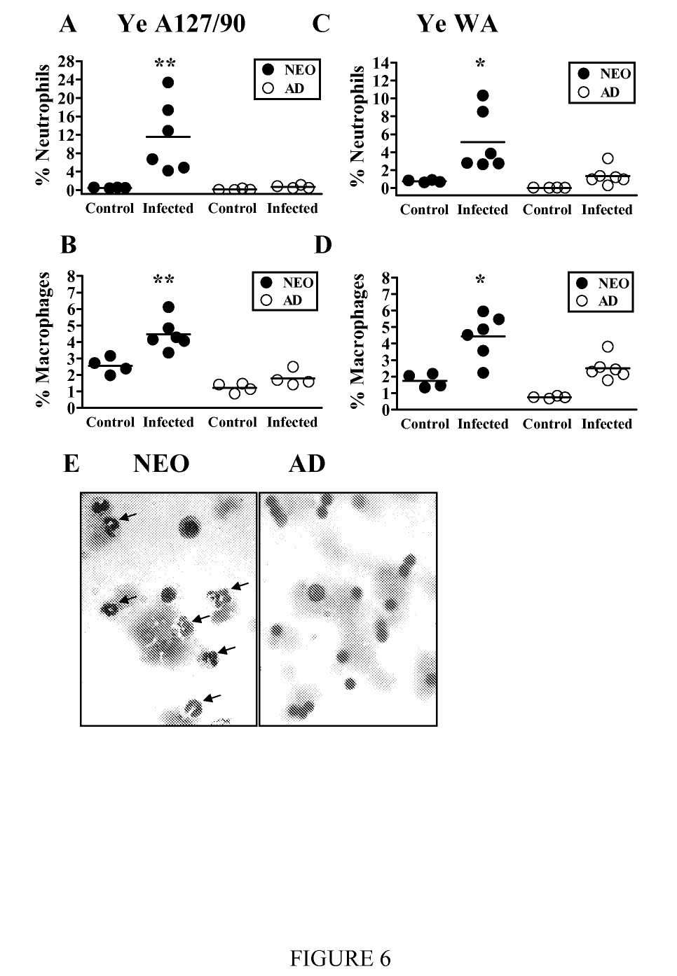

Following o.g. infection in neonates, there is a marked influx of innate phagocytes into the neonatal MLN. The observation that the majority of Y. enterocolitica organisms are contained in the intestine and MLN and do not reach peripheral tissues is consistent with the idea that the infection may be controlled by regional innate immune responses in the neonatal gut. A substantial difference between neonates and adults infected with a lethal dose is in the extent of gross tissue changes that occur in the MLN. Indeed, it was observed that the neonatal MLN increase dramatically in size and weight (>2- to 11-fold increase in weight compared with those of age-matched uninfected controls) by 7 days after o.g. infection with 5×10⁷ CFU Y. enterocolitica (FIG. 5A). Adults infected with the same dose showed little to no change in MLN weight (≧1.8-fold increase) (FIG. 5A). Based on the pattern of bacterial colonization and the changes in organ size, it was hypothesized that resistance in neonates may be attributed to enhanced recruitment of innate phagocytes that function to limit the spread of the bacteria beyond the neonatal gut. To test this idea, the influx of neutrophils and macrophages, two innate phagocytes which have been shown to be important in controlling Y. enterocolitica infection in adult mice, was analyzed. Flow cytometric analysis of individual MLN of neonates and adult mice infected o.g. with 5×10⁷ CFU Y. enterocolitica A127/90 was conducted. Neutrophils were identified as Gr-1hi CD68int cells, and macrophages were identified as Gr-1lo-int CD68hi cells (FIG. 5B). There was an increase in the percentages of both neutrophils (FIG. 6A) and macrophages (FIG. 6B) in the MLN of most infected neonates analyzed as early as 3 days p.i. compared with age-matched uninfected controls. However, there were no significant changes in the percentages of neutrophils (FIG. 6A) and macrophages (FIG. 6B) in the adult MLN analyzed at 3 (FIG. 6) and 5 days p.i.

Significant differences (P=0.0095) were observed in the percentages of neutrophils (16-fold) and macrophages (2.5-fold) in the MLN of infected neonates compared to those in the MLN of infected adults. The limited phagocyte infiltration in the adult MLN could not be accounted for solely by the absence of Y. enterocolitica in this organ because there were similar mean titers (mean ± standard deviation) at 4 days p.i. in the MLN of neonates (6.1±2.1) and adults (7.7±0.3) infected with this dose. In addition, the low percentages of neutrophils and macrophages in the adult MLN could not be explained as a failure to detect these cell populations because staining of PP and spleen cells from the same mice revealed increases in both populations as early as 3 days p.i., which is consistent with previous reports. These results indicated that even at very high doses of bacteria (FIG. 4A), inflammatory infiltration into the adult MLN was decreased compared to that for neonates. To determine that these results were not limited to the A127/90 strain, neutrophils and macrophages infiltrating the neonatal MLN after infection with 5×10⁸ CFU of Y. enterocolitica strain WA were measured. Similar to what was observed with a high dose of Y. enterocolitica A127/90, significantly greater percentages of neutrophils (3.8-fold; P=0.0152) (FIG. 6C) and macrophages (1.8-fold; P=0.0411) (FIG. 6D) were observed in infected neonates than in infected adult mice. To confirm that the large influx observed in neonates by flow cytometry was indeed composed mostly of neutrophils, Wright-Giemsa staining of MLN cells from the same mice was performed (FIG. 6E). Using this method, percentages of neutrophils comparable to those observed by flow cytometry were found for the MLN of uninfected and infected neonatal and adult mice (Table 3). The differences in percentages of neutrophils in the neonatal MLN compared to adult mice infected with Y. enterocolitica A127/90 and WA were 17- and 10-fold, respectively. Altogether, these results indicate that neonatal mice have a proportionally greater neutrophil infiltration than do adult mice infected with two different high-virulence Y. enterocolitica strains.

TABLE 3
Neutrophils in MLN infected with Y. enterocolitica strain

| | A127/90 | | WA | |
|---|---|---|---|---|
| Group | Mean (%)a | Range (%) | Mean (%)a | Range (%) |
| Uninfected | 0.97 | 0.22-2.02 | 0.92 | 0.76-1.14 |
| neonates | | | | |
| Infected | 14.74^{b,c} | 0.97-36.53 | 10.02^{b,c} | 1.04-19.36 |
| neonates | | | | |
| Uninfected | 0.08 | 0.00-0.24 | 0.02 | 0.00-0.09 |
| Adults | | | | |
| Infected | 0.87^{d} | 0.15-1.42 | 1.00^{d} | 0.64-2.27 |
| Adults | | | | |

apercentage of neutrophils analyzed by Wright-Giemsa staining 3 days after infection with 5 × 10⁷ CFU Y. enterocolitica A127/90 or 5 × 10⁸ CFU Y. enterocolitica WA. Three or four uninfected and four to six infected mice were analyzed per group.
^{b}P ≦ 0.024 compared to uninfected neonates by Mann-Whitney test.
^{c}P ≦ 0.004 compared to infected adults by Mann-Whitney test.
^{d}P ≦ 0.017 compared to uninfected adults by Mann-Whitney test.

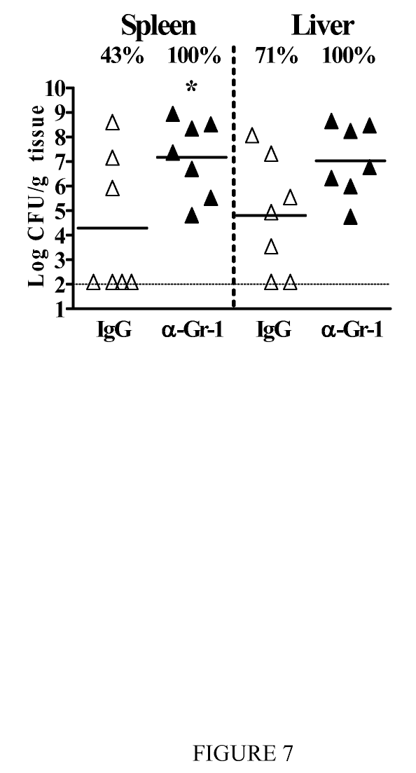

Neutrophil depletion leads to a greater proportion of neonates with high bacterial titers in the spleen and liver. Since neutrophils are highly phagocytic, the increased neutrophil infiltration in the neonatal MLN suggested that this cell population in particular may contribute to the resistance of neonates after o.g. infection. In this case, neutrophils would be important in controlling bacterial replication in neonatal intestinal tissues. To address this possibility, the impact of neutrophil depletion prior to infection with a high bacterial dose was analyzed. The monoclonal antibody RBC-8C5 (anti-Gr-1), which selectively targets neutrophils and has previously been used successfully in neonatal mice, was used. To verify that neutrophils were depleted by this antibody under the infection conditions, flow cytometric analysis of cells from infected mice determined that Gr-1hi CD68int cells were reduced to <0.02% of cells in the neonatal MLN and spleen after injection with the anti-Gr-1 antibody. Therefore, it was assumed that the antibody treatment effectively reduced the neutrophil population. Neonatal mice were treated with anti-Gr-1 antibody 1 day prior to and 1 day following infection with 10× LD₅₀, and their tissues were analyzed 8 days p.i. for bacterial colonization. The bacterial titers found in the intestines and MLN did not differ between groups, indicating that all mice were productively infected. However, the anti-Gr-1 antibody treatment increased the proportion of neonatal mice with detectable Y. enterocolitica to 100% for the spleen and liver, in contrast to 43% and 71% for the control group (FIG. 7). The mean bacterial titers in the neutrophil-depleted neonates for both organs were at least 2 log higher than those found in the control mice, although statistical significance (P=0.038) was only evident for the spleen. These results suggest that in the absence of neutrophils, the bacteria are able to replicate and disseminate into peripheral tissues at greater rates, resembling the colonization pattern observed in adults. These observations indicate that neonatal neutrophils may contribute substantially to the increased resistance observed in neonates. The combined results from these experiments support the hypothesis that the innate immune system of neonates rapidly mobilizes phagocytes to the gut and that these phagocytes efficiently protect against Y. enterocolitica introduced through the natural route of infection.

### Example 2

### Neonatal Yersinia enterocolitica Infection

Classically, neonates have been considered not competent to respond to infectious agents. Several quantitative and qualitative differences in innate and adaptive immune responses compared to adults exist, including: neonates have reduced numbers of immune cells; neonates have reduced pro-inflammatory cytokine and chemokine production; and neonates have an impaired innate phagocyte function. A murine neonatal model of Y. enterocolitica orogastric infection is useful because two-thirds of Y. enterocolitica cases are diagnosed in children under the age of 4 and the pathology is similar to human disease, making this a highly relevant pediatric model for gastroenteric disease. A murine neonatal model to study mucosal immune responses to an extracellular enteric bacterial pathogen was heretofore unknown.

There are several pathogenic Yersinia species, including Y. pestis, Y. pseudotuberculosis and Y. enterocolitica. Y. pseudotuberculosis and Y. enterocolitica are food borne pathogens. These bacteria replicate in Peyer's patches and mesenteric lymph nodes (MLN), and can cause enterocolitis, mesenteric lymphadenitis, and cases of septicemia. Such an infection can be misdiagnosed as appendicitis.

In the murine adult model of Y. enterocolitica infection, clearance of the pathogen requires Th1 immunity (IFN-γ, IL-12, IL-18 and TNF-α) which promotes phagocytic function and macrophage activation. In addition, IL-17 mRNA has been detected in the PP and MLN of infected adult mice, but there is no direct evidence for its requirement in protection. Finally, antibodies generated during an infection are protective against re-exposure.

Under most immunization conditions, neonatal primary responses are mixed; Both IFNγ (Th1 cytokine) and IL-4 (Th2 cytokine) are detectable. However they have been shown to have compromised development of memory Th1 responses and reduced neutralizing antibody responses. Although, protective responses can be generated in neonatal mice by using agents that induce strong Th1 responses, e.g. DNA vaccines, CpG oligonucleotides, and bacterial adjuvants (e.g., E. coli labile toxin).

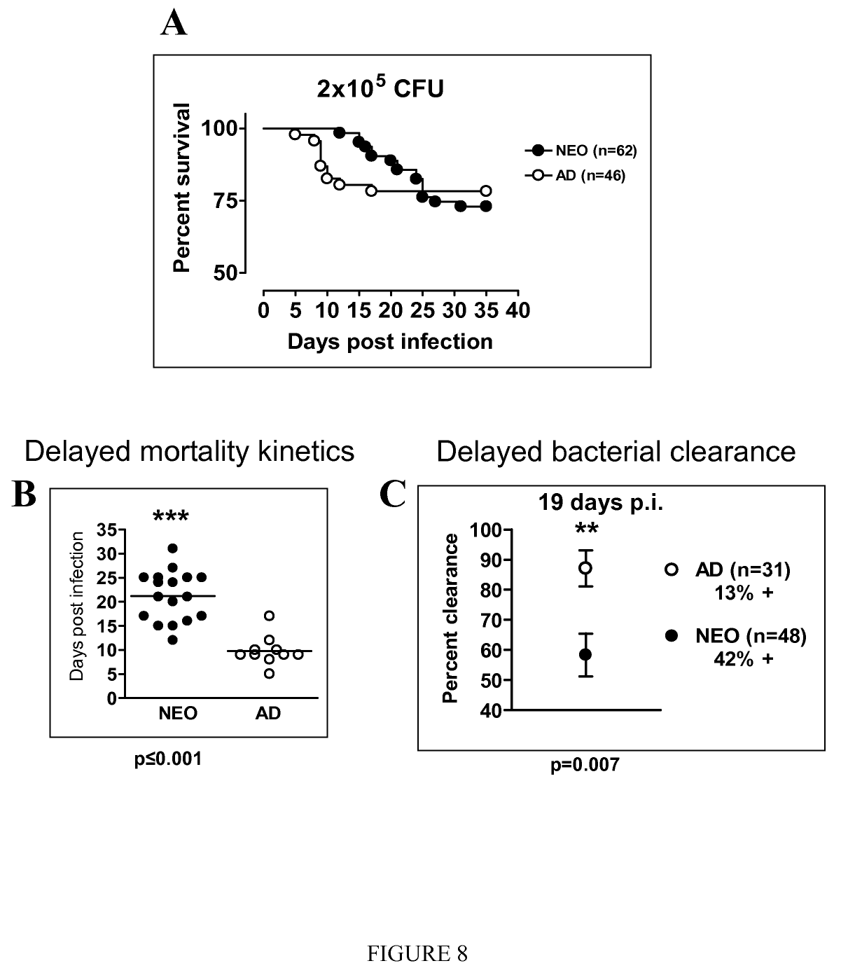

FIG. 8 demonstrated that neonatal mice have prolonged survival when infected with a low bacterial dose (0.1× LD₅₀) and that the onset of mortalities is delayed compared to adult mice. In addition, a greater proportion of neonatal mice harbored the bacteria for longer period of time compared to adult mice. This suggests that neonatal mice might be capable of mounting a protective adaptive immune response.

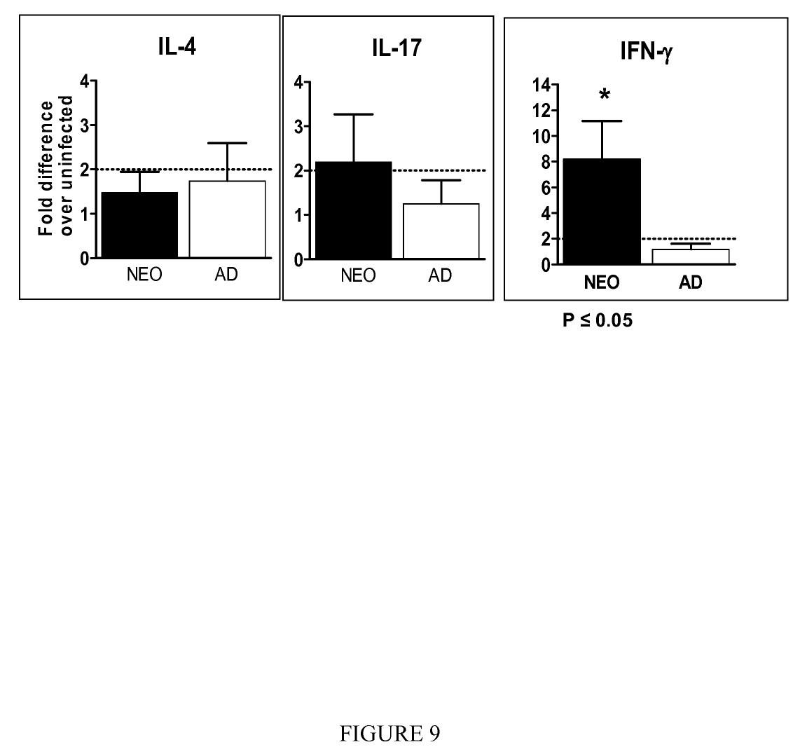

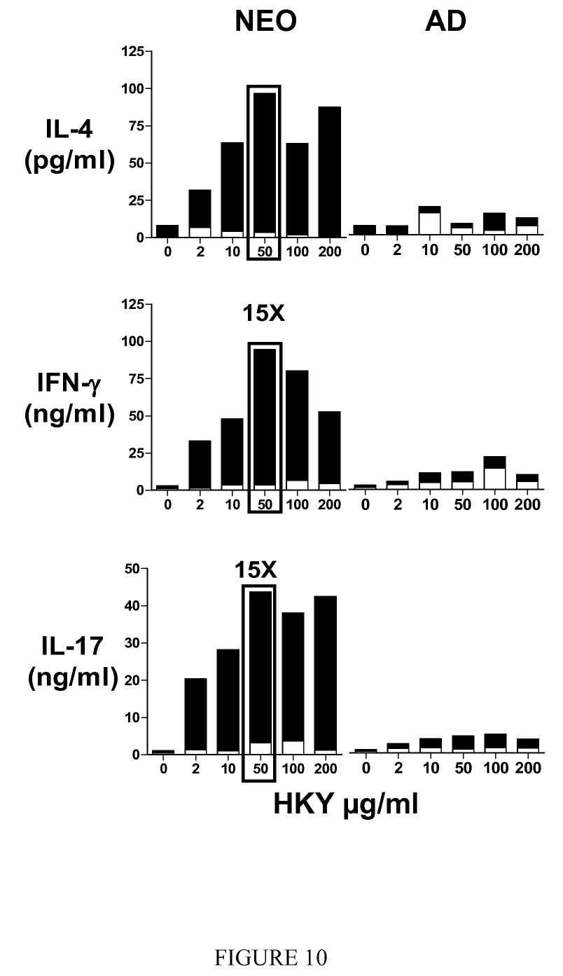

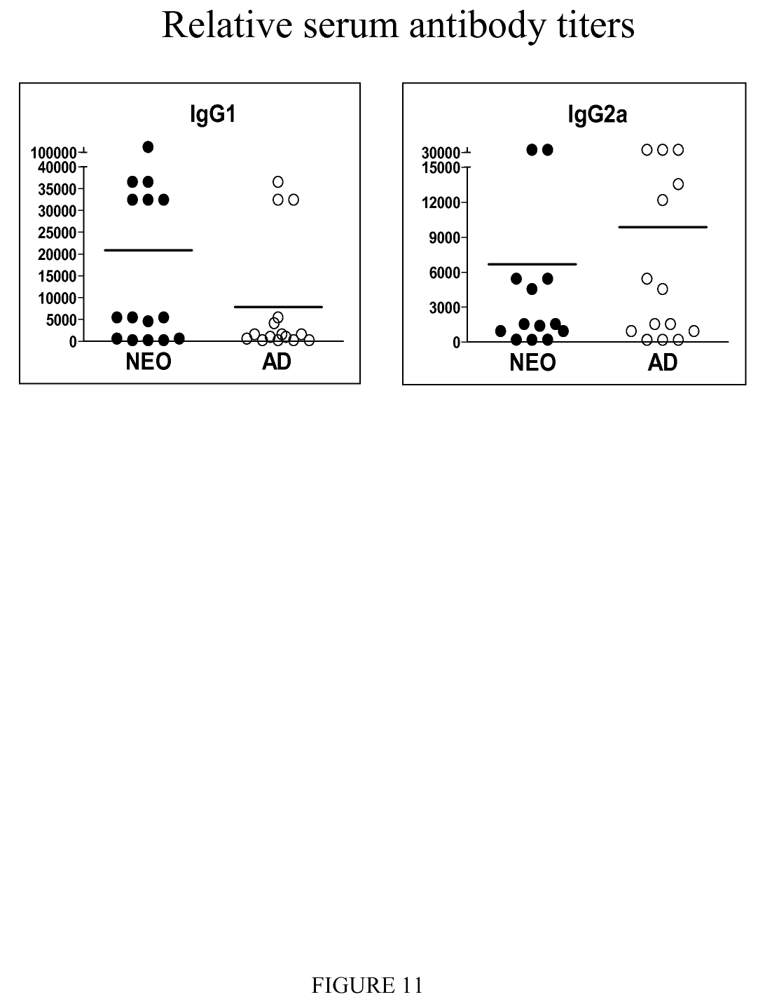

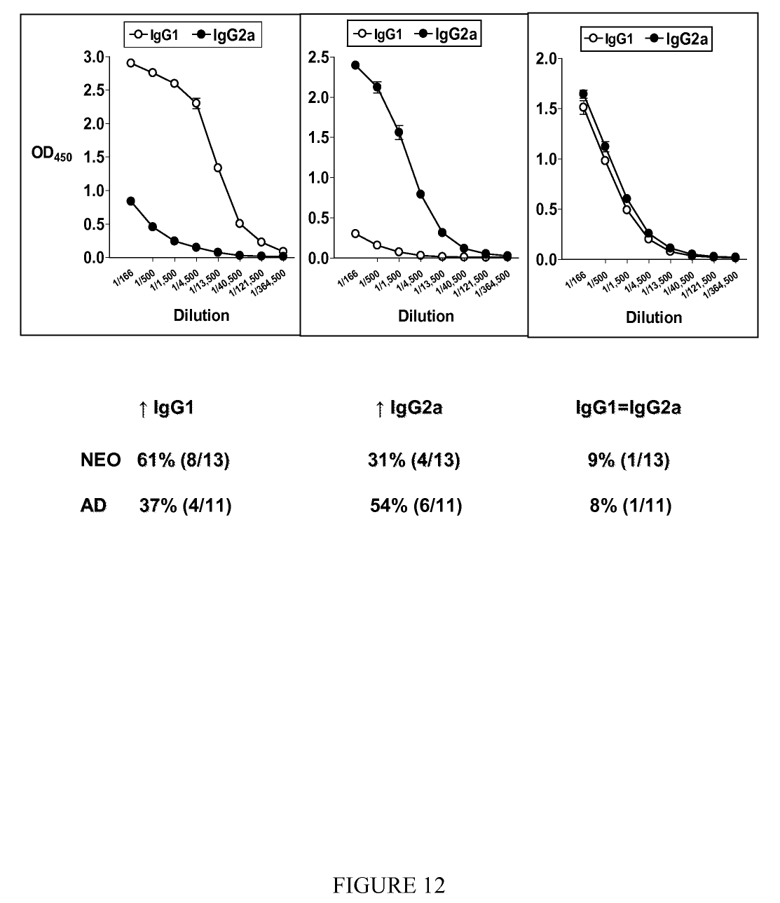

Whether or not pro-inflammatory cytokines are detectable in the MLN was examined. Increased IFN-γ mRNA was detected in the MLN of neonatal mice infected with a low bacterial dose (see FIG. 9). Referring to FIG. 10, neonatal mice have a mixed primary CD4 T cell response in the MLN. Neonatal mice mount robust IFN-γ and IL-17 responses compared to adult mice. Antibody responses of neonatal and adult mice to a secondary infection in adulthood were examined. Neonatal and adult mice had comparable memory antibody levels when boosted in adulthood (FIG. 11). However, the pattern of secondary antibody responses was heterogeneous in mice boosted in adulthood (FIG. 12). There are a greater proportion of neonatal mice with an IgG1-predominant response.

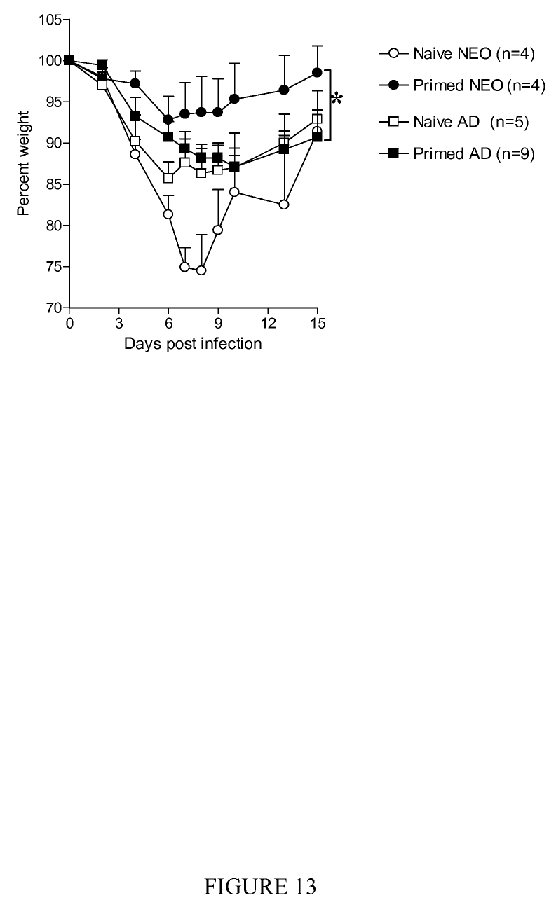

Whether or not mice are protected against a lethal challenge was examined. Mice initially infected as neonates show enhanced protection against a bacterial challenge (see FIG. 13).

The results described herein support several conclusions. Inflammatory T cell responses are generated during the primary response (mixed Th1-Th17). Therefore, neonatal T cells might be able to stimulate and amplify the initial innate immune response. Yersinia-specific memory antibody responses are generated in both neonatal and adult mice. Mice can mount both Th1 and Th2 associated antibody responses—the relative levels are similar between neonates and adults. The proportion of neonatal mice with a Th2 associated response is increased (60% vs 40% of mice). In addition, mice initially infected as neonates might be better protected against a lethal challenge in adulthood.

The mechanism by which Yersinia species infect cells involves several steps and components of the Yersinia machinery. Contact of Yersinia with host cells promotes delivery of effector proteins called “Yops” through a Type III Secretion System (YopO/YpkA, YopT, YopE, YopH, YopM, YopP). Some of the Yersinia Yops block phagocytosis by disorganizing the host cytoskeleton and depolymerizing the actin microfilaments. However, YopP has been shown to block an inflammatory response by reducing cytokine and chemokine production by targeting both the MAPK and NFκB pathways. YopP has also been shown to induce cell death in macrophages and dendritic cells. These characteristics placed YopP as an important mediator in the inflammatory response observed during neonatal infection.

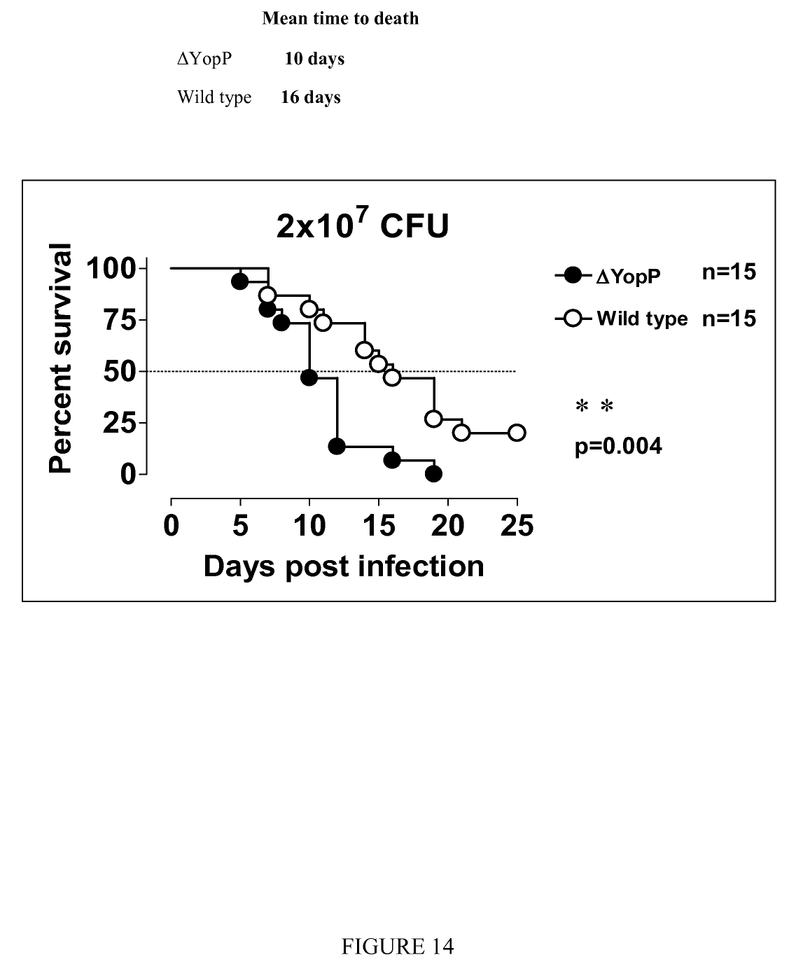

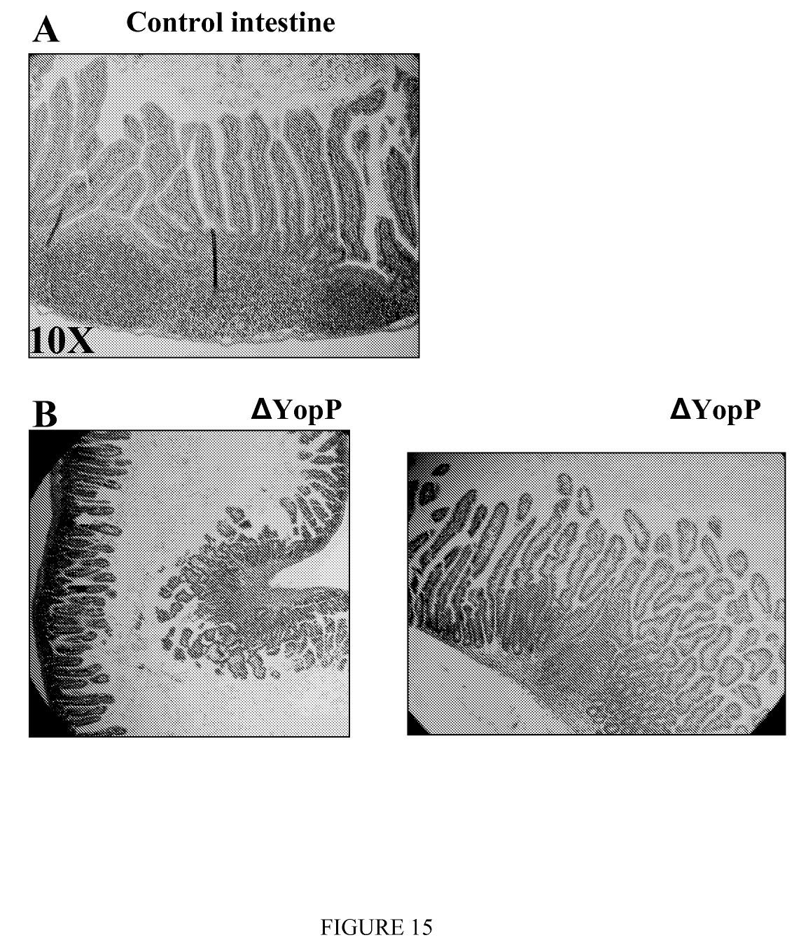

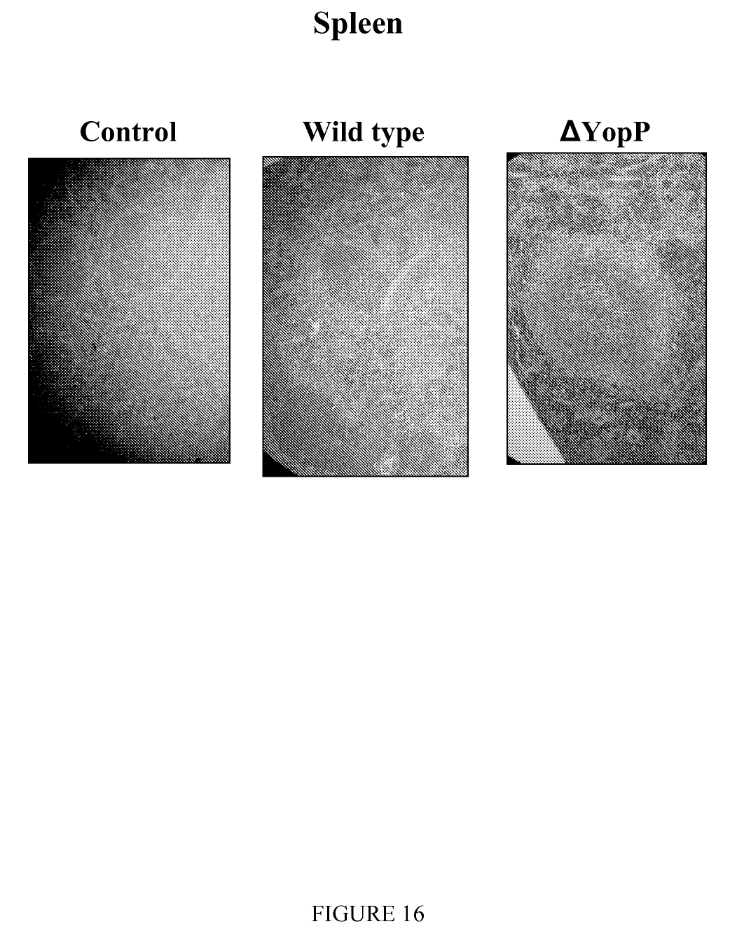

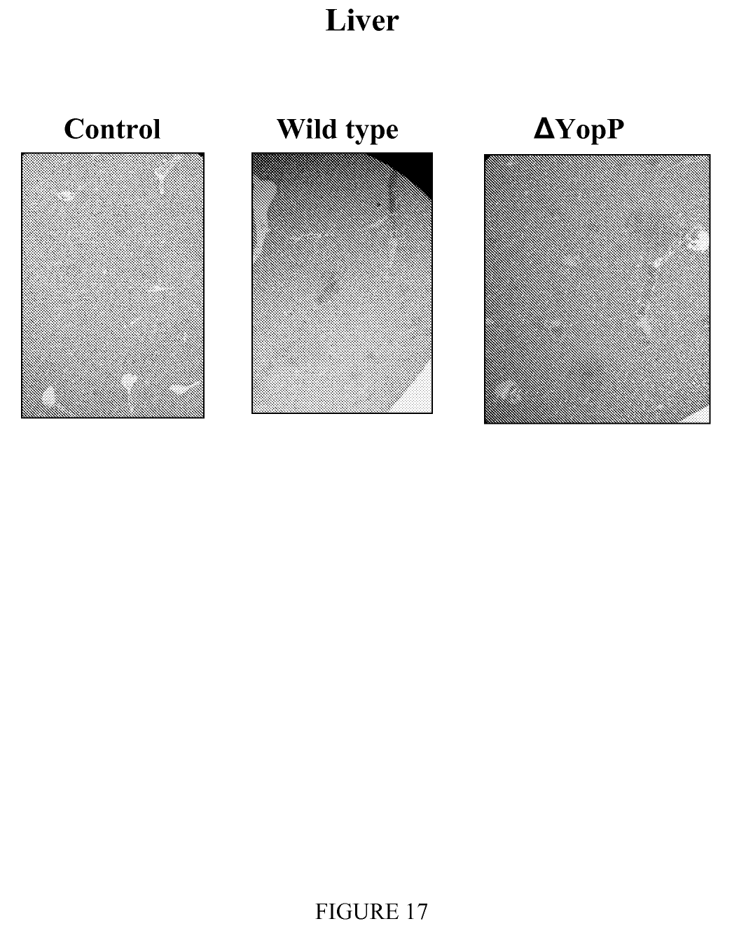

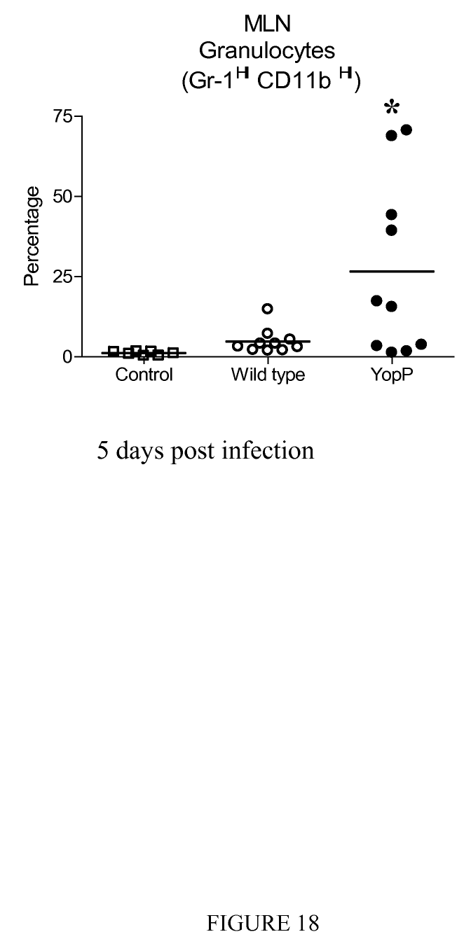

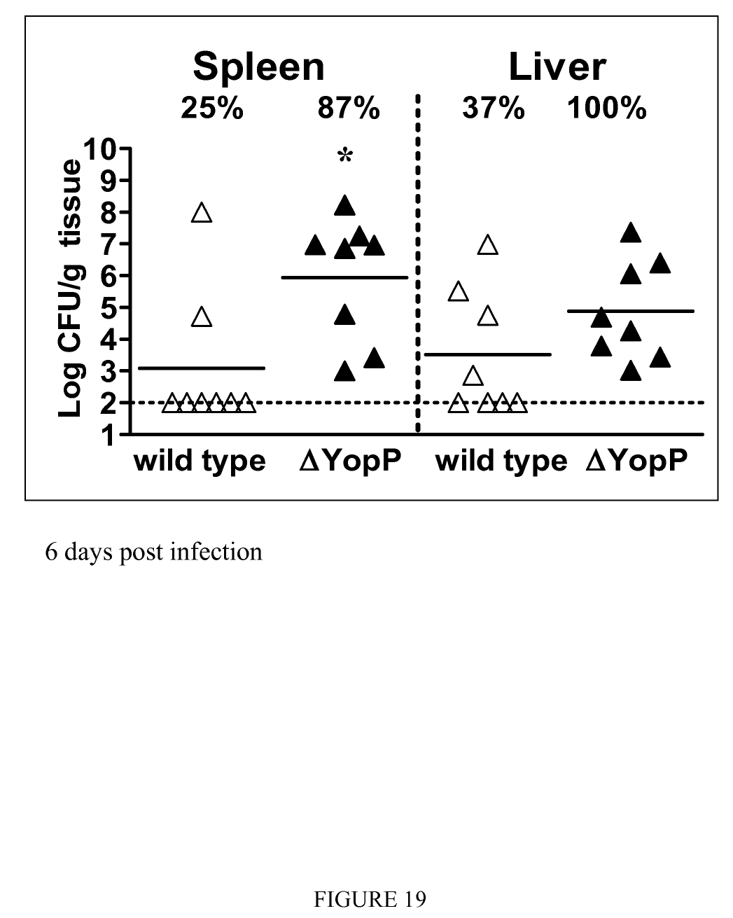

To investigate the role of YopP during infection in neonates, we compared multiple parameterd following infection with the wild type strain and mutant strain lacking yopP (ΔYopP). Referring to FIG. 14, in neonatal mice, infection with a ΔYopP mutant leads to faster death kinetics. FIG. 15 depict profound damage to the intestinal tissue of ΔYopP infected neonates. Neonates infected with the ΔYopP mutant show inflammation of peripheral tissues like the spleen (FIG. 16) and liver (FIG. 17). Referring to FIG. 18, phagocytes infiltrated to a greater level the MLN of neonates infected with the ΔYopP mutant. Referring to FIG. 19, despite a greater mobilization of innate cells, there was increased bacterimia in neonates infected with the YopP mutant.

In conclusion, a Yersinia protein (YopP) was identified which appears to improve the survival of neonatal mice, suggesting that YopP might function as a protective factor, rather than a virulence factor, in the developing neonate. In a working model of a wild-type infection, innate immune cell infiltration into intestinal tissues is protective, resulting in reduced bacterimia into peripheral tissues and reduced global inflammation. In a working model of a ΔYopP infection, a more robust innate immune cell infiltration into intestinal tissues causes damage, resulting in increased bacterimia into peripheral tissues and more inflammation in the periphery.

### Other Embodiments

Any improvement may be made in part or all of the compositions and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A composition comprising an immunologically effective amount of at least one Yop protein or a nucleic acid encoding at least one Yop protein and a pharmaceutically acceptable diluent or carrier.

2. The composition of claim 1, wherein the at least one Yop protein or nucleic acid encoding at least one Yop protein is an adjuvant.

3. The composition of claim 2, further comprising a second adjuvant.

4. The composition of claim 1, wherein the Yop protein is a Yersinia enterocolitica Yop protein.

5. A method comprising administering an immunologically effective amount of a composition comprising at least one Yop protein or a nucleic acid encoding at least one Yop protein and a pharmaceutically acceptable diluent or carrier to a neonate or child that results in promotion of an immune response in the neonate or child.

6. The method of claim 5, wherein the composition is administered mucosally.

7. The method of claim 5, wherein the neonate or child is suffering from a bacterial gastroenteric infection.

8. The method of claim 5, wherein the at least one Yop protein or nucleic acid encoding at least one Yop protein is an adjuvant.

9. The method of claim 8, wherein the composition further comprises a second adjuvant.

10. The method of claim 5, wherein the Yop protein is a Yersinia enterocolitica Yop protein.

11. A method comprising administering a therapeutically effective amount of a composition comprising at least one Yop protein or a nucleic acid encoding at least one Yop protein and a pharmaceutically acceptable diluent or carrier to a neonate or child suffering from an autoimmune disease that results in decreasing or preventing inflammation in the neonate or child.

12. The method of claim 5, wherein the composition is administered mucosally.

13. The method of claim 5, wherein the at least one Yop protein or nucleic acid encoding at least one Yop protein is an adjuvant.

14. The method of claim 13, wherein the composition further comprises a second adjuvant.

15. The method of claim 5, wherein the Yop protein is a Yersinia enterocolitica Yop protein.
